(19) United States
(12) Patent Application Publication
(10) Pub. No.: US 2007/0106238 A1
(43) Pub. Date: May 10, 2007

(54) Waste collection system

(75) Inventors: David Groll, Murrysville, PA (US); Mark Kaforey, Murrysville, PA (US); Craig Kaforey, Oakmont, PA (US)

Correspondence Address: JAMES RAY & ASSAOCIATES, 2640 PITCAIRN ROAD, MONROEVILLE, PA, 15146, US

(73) Assignee: XODUS MEDICAL INC.

(21) Appl. No.: 11/268,820

(22) Filed: Nov. 8, 2005

(51) Int. Cl. A61M 1/00

(52) U.S. Cl. 604/323

(57) ABSTRACT

A waste collection system for the collection and containment of contaminated fluids comprises a receptacle having at least one sidewall, a bottom portion and an open top portion. A containment means engaged with the receptacle for sealing the open top portion of the receptacle in a fluid tight manner and for retaining the contaminated fluids within the receptacle. A valve means associated with the containment means. The valve means extending a predetermined distance below the plane of the containment means. An aperture extending through the valve means. This aperture having a size and shape capable of allowing a fluid deposit member to extend there through. A fluid directing means extending from the aperture into the receptacle for directing contaminated fluid deposited therein away from the aperture and into the receptacle.

### FIELD OF THE INVENTION

The present invention relates, in general, to a medical waste collection and disposal system, and more particularly, to a system for effectively collecting, containing, and disposing contaminated medical fluids.

### BACKGROUND OF THE INVENTION

The collection, containment, and disposal of contaminated medical or physiological fluids has long been a concern in the waste disposal industry. These contaminated fluids often contain excess medications, blood and diseased fluids including HIV (AIDS), hepatitis, and other communicable diseases. Current procedures for collecting these fluids, such as during angioplasty procedures or catheterizations, include the use of open waste basins, towels, sponges and vinyl film bag/tubing systems. These techniques pose a significant health risk to medical personnel. Furthermore, if not properly disposed, these fluids can pose an environmental hazard as well.

U.S. Pat. No. 5,483,999 is directed to a waste collection system for containment and disposal of contaminated fluids. The waste collection system comprises a box shaped receptacle containing an absorbent pad therein and a molded plastic containment lid, which is fitted onto the receptacle in a fluid tight manner. The lid includes an opening within which the contaminated fluids may be deposited into the receptacle. A circular disc constructed of a foam material is placed in the opening. The foam material of the circular disc provides absorption and wicking action for fluids contacting the foam materials so that the fluids will readily pass through the foam material without splashing and without beading up. The circular disc is also provided with a number of slits so that fluid can readily be injected, for example, by means of a syringe by sticking the end of the syringe through the slits formed in the disc and depositing the contents onto the absorbent pad contained within the receptacle. The collection system can also include an additional lid which may be sealed onto the receptacle prior to disposal.

The waste collection system of U.S. Pat. No. 5,483,999 is a vast improvement of many of the techniques previously used for collecting medical/physiological fluids, however medical personnel are still at risk of exposure to these contaminated fluids. A major drawback in the ('999) system is that the foam material, which comes into contact with the fluids, is exposed. Consequently, medical personnel run the risk of contacting this contaminated foam material with their hand, arm or other part of their body. Also, since this foam material is in the same plane as the bottom portion of the lid, some of the contaminated fluid may “pool” and/or remain on the surface of the lid, once again risking unwanted exposure to medical personnel. Thirdly, should the medical person depositing the waste material fail to fully insert the syringe containing the contaminated fluid through the slits in the foam, then this fluid would go into the foam. While the ('999) patent relies upon wicking for the fluid to fall into the receptacle this wicking action takes time and thus the possibility of exposure to a greater amount of contaminated fluid exists.

Objects of the Invention

It is therefore an object of the invention to provide a waste collection system which draws contaminated fluid away from the opening to reduce exposure to the fluid by medical personnel.

It is still another object of the invention to provide a protective cover over the valve means to further reduce exposure to the fluid by medical personnel.

It is a further object of the invention to provide a waste collection system which effectively contains contaminated fluid for environmentally responsible disposal thereof. In addition to the above-described objects and advantages of the waste collection system, various other objects and advantages of the present invention will become more readily apparent to the persons who are skilled in the same and related arts from the following more detailed description of the invention, particularly, when such description is taken in conjunction with the attached drawing figures and the appended claims.

### SUMMARY OF THE INVENTION

Briefly, and in accordance with the forgoing objects, the present invention is directed to a waste collection system for the collection and containment of contaminated fluids that overcomes the problems associated with previously used waste collection systems. The waste collection system of the present invention comprises a receptacle for receiving contaminated fluids therein. The receptacle has at least one sidewall, a bottom portion and an open top portion. A containment means is engaged with the receptacle for sealing the open top portion of the receptacle in a fluid tight manner and for retaining the contaminated fluids within the receptacle. A valve means is associated with the containment means. The valve means extends a predetermined distance below the plane of the containment means. An aperture extends through the valve means. This aperture has a size and shape capable of allowing a fluid deposit member to extend there through. A fluid directing means is provided which extends from the aperture into the receptacle for directing contaminated fluid deposited therein away from the aperture and into the receptacle.

### BRIEF DESCRIPTION OF THE DRAWINGS

FIG. 1 shows a perspective view of the waste disposal system of the invention.

FIG. 2 shows a top view of the waste disposal system of FIG. 1.

FIG. 3 shows a side view of the waste disposal system taken along line A-A of FIG. 2.

FIG. 4 shows an enlarged view of the valve means shown in FIG. 3.

FIG. 5 shows an enlarged view of the hinge means for attaching the lid to the receptacle/containment means of the waste disposal system of FIG. 1.

FIG. 6 shows a perspective view of the waste disposal system according to another embodiment of the invention.

### DETAILED DESCRIPTION OF THE INVENTION

Before describing the invention in detail, the reader is advised that, for the sake of clarity and understanding, identical components having identical functions have been marked where possible with the same reference numerals in each of the Figures provided in this document.

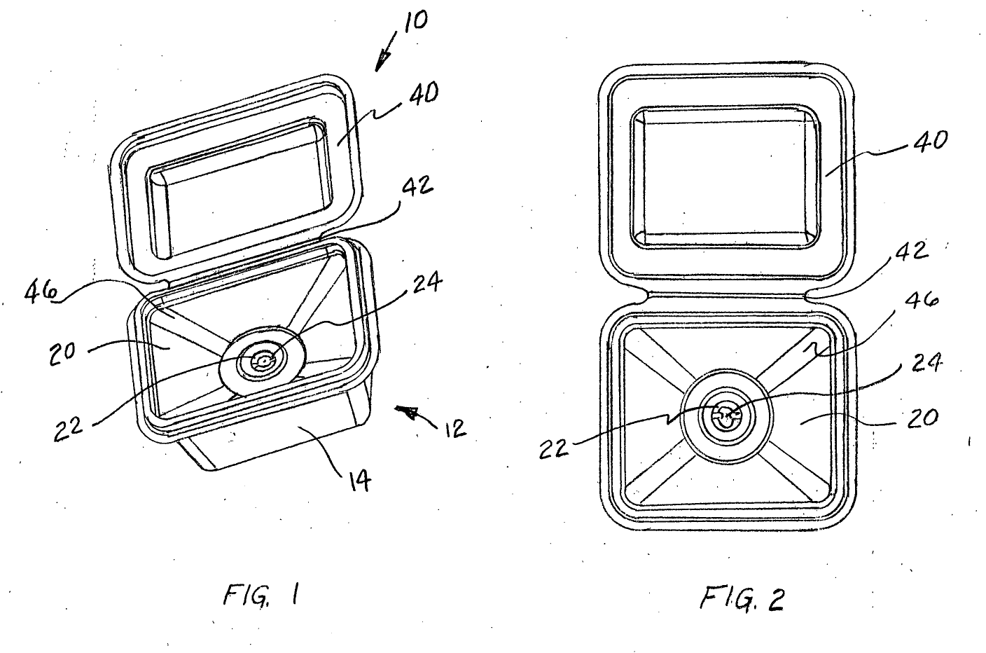

Referring now to FIGS. 1-3 there is shown the waste disposal system of the invention, generally indicated as 10. The waste disposal system comprises a receptacle 12 for receiving contaminated fluids therein. The receptacle has at least one sidewall 14, a bottom portion 16, and an open top portion 18. A containment means 20 is engaged with the receptacle 12 for sealing the open top portion 18 of the receptacle 12 in a fluid tight manner and for retaining the contaminated fluids within the receptacle 12. A valve means 22 is associated with the containment means. The valve means 22 extends a predetermined distance below the plane of the containment means 20 to reduce “pooling” of the contaminated fluid on the containment means 20. An aperture 24 extends through the valve means 22. This aperture 24 has a size and shape capable of allowing a fluid deposit member 26 to extend there through. A fluid directing means 28 is provided which extends from the aperture 24 into the receptacle 12 for directing contaminated fluid deposited therein away from the aperture 24 and down into the receptacle 12.

The receptacle 12 may be in the shape of a box having four sidewalls 14, a bottom portion 16 and an open top portion 18. The containment means 20 will then have a predetermined size of at least one of a square and a rectangle which is capable of engaging with and sealing the open top portion 18 of the receptacle 12.

In an alternative design, as illustrated in FIG. 6, the receptacle 12 may be cylindrical in shape having a cylindrical sidewall 14, a round and/or ovular bottom portion 16 and a round and/or ovular open top portion 18. The containment means 20 will have a predetermined size of at least one of a circle and an oval which is capable of engaging with and sealing the open top portion 18 of the receptacle 12.

Preferably, the receptacle 12 is a molded plastic article having a snap ring 50 formed as a rim around the open top portion 18. The containment means 20 is a molded plastic article having a shoulder 52 molded around its periphery thereof. This shoulder 52 is capable of cooperating with the snap ring so as to form a fluid tight seal between the receptacle 12 and the containment means 20.

The containment means 20 includes at least one sloped portion 30 that slopes downwardly to meet a top edge portion 32 of the valve means 22. Preferably, the containment means 20 includes multiple sloped portions that slope downwardly to meet at the top edge portion 32 of the valve means 22.

The sloped portions 30 include a channel 46 having end portion 48 which runs underneath the valve means 22 so that any contaminated fluids deposited on the containment means run down the channel 46, under the valve means 22, and into the receptacle 12.

The valve means 22 includes an absorbent material 48 around its edges to absorb contaminated fluids which run down the channel 46 and underneath the valve means 22.

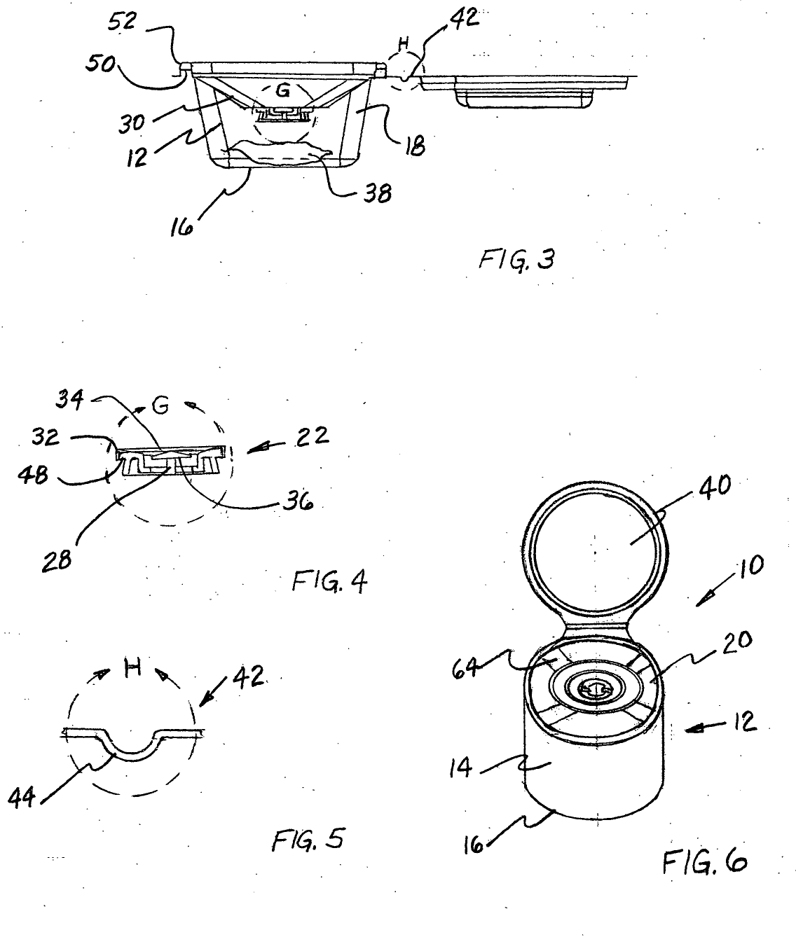

As illustrated in FIGS. 3 and 4, the valve means 22 includes a protective top cover 34 and the aperture 24 extends through the protective top cover 34. The protective top cover 34 is impermeable to prevent splash back from contaminated fluid being deposited within the receptacle 12.

The valve means 22 includes a first absorbent member 36 positioned between the protective top cover 34 and the fluid directing means 28 for absorbing any residual contaminated fluid which is deposited into the valve means 22.

As illustrated in FIG. 3, a second absorbent member 38 is positioned adjacent the bottom portion 16 of the receptacle. This second absorbent member 38 may comprise one of a sponge, an absorbent pad, loose absorbent material, and the like.

A lid 40 is provided which is capable of being attached to containment means/receptacle for disposal of the waste containment system. The lid 40 includes an attachment means 42 which is attached to either the containment means 20 or the sidewall 14 of the receptacle 12. As illustrated in FIG. 5, the attachment means 42 includes at least one folding member 44 which is molded into the attachment means 42 to allow the lid 40 to flip open and closed. The lid 40 is preferably formed of a molded plastic material and is capable of forming a fluid tight seal with the containment means/receptacle so that the waste collection system can be disposed of in an environmentally responsible manner.

The invention has been described in such full, clear, concise, and exact terms so as to enable any person skilled in the art to which it pertains to make and use the same. It should be understood that variations, modifications, equivalents and substitutions for components of the specifically described embodiments of the invention may be made by those skilled in the art without departing from the spirit and scope of the invention as set forth in the appended claims. Persons who possess such skill will also recognize that the foregoing description is merely illustrative and not intended to limit any of the ensuing claims to any particular narrow interpretation.

We claim:

1. A waste collection system for the collection and containment of contaminated fluids, said waste collection system comprising:
(a) a receptacle for receiving contaminated fluids therein, said receptacle having at least one sidewall, a bottom portion and an open top portion;
(b) a containment means having a predetermined size and shape engaged with said receptacle for sealing said open top portion of said receptacle and for retaining such contaminated fluids within said receptacle;
(c) valve means associated with said containment means, said valve means extending a predetermined distance below the plane of said containment means;
(d) an aperture extending through said valve means, said aperture having a size and shape capable of allowing a fluid deposit member to extend there through; and
(e) a fluid directing means extending from said aperture into said receptacle for directing contaminated fluid deposited therein away from said aperture and into said receptacle.

2. A waste collection system as recited in claim 1 wherein said receptacle is in the shape of a box having four sidewalls, a bottom portion and an open top portion.

3. A waste collection system as recited in claim 2 wherein said containment means has a predetermined size of at least one of a square and a rectangle and is capable of engaging with and sealing said open top portion of said receptacle.

4. A waste collection system as recited in claim 1 wherein said receptacle is cylindrical in shape having a cylindrical sidewall, a bottom portion and an open top portion.

5. A waste collection system as recited in claim 4 wherein said containment means has a predetermined size of at least one of a circle and an oval and is capable of engaging with and sealing said open top portion of said receptacle.

6. A waste collection system as recited in claim 1 wherein said containment means includes at least one sloped portion that slopes downwardly to meet a top edge portion of said valve means.

7. A waste collection system as recited in claim 6 wherein said containment means includes multiple sloped portions that slope downwardly to meet at said top edge portion of said valve means.

8. A waste collection system as recited in claim 1 wherein said valve means includes a protective top cover and wherein said aperture extends through said protective top cover.

9. A waste collection system as recited in claim 8 wherein said protective top cover is impermeable to prevent splash back from contaminated fluid being deposited within said receptacle.

10. A waste collection system as recited in claim 8 wherein said valve means includes a first absorbent member positioned between said protective top cover and said fluid directing means.

11. A waste collection system as recited in claim 1 further including a second absorbent member positioned adjacent said bottom portion of said receptacle.

12. A waste collection system as recited in claim 11 wherein said second absorbent member comprises one of a sponge, an absorbent pad, and loose absorbent material.

13. A waste collection system as recited in claim 1 further including a lid capable of being attached to said containment means.

14. A waste collection system as recited in claim 13 wherein said lid includes an attachment means at one end and wherein said attachment means is attached to one of said containment means and said at least one sidewall of said receptacle.

15. A waste collection system as recited in claim 14 wherein said attachment means includes at least one folding member to allow said lid to flip open and closed.

16. A waste collection system as recited in claim 6 wherein said at least one sloped portion includes a channel, said channel having an end portion which runs underneath said valve means so that any contaminated fluids deposited on said containment means run down said channel, under said valve means, and into said receptacle.

17. A waste collection system as recited in claim 16 wherein said valve means includes an absorbent material around its edges to absorb contaminated fluids.

18. A waste collection system as recited in claim 1 wherein said receptacle is a molded plastic article having a snap ring formed as a rim around said open top portion.

19. A waste collection system as recited in claim 18 wherein said containment means is a molded plastic article having a shoulder molded around its periphery thereof, said shoulder capable of cooperating with said snap ring so as to form a fluid tight seal between said receptacle and said containment means.
